(19) United States
(12) Patent Application Publication
(10) Pub. No.: US 2026/0041900 A1
(43) Pub. Date: Feb. 12, 2026

(54) NEEDLE-BASED AUTO-INJECTOR PORT ADAPTER

(71) Applicant: RX BANDZ, INC., Locust Valley, NY (US)

(72) Inventors: Grant Miller, Washington, NJ (US); Stephen Harhen, Rumson, NJ (US)

(21) Appl. No.: 19/292,999

(22) Filed: Aug. 7, 2025

Related U.S. Application Data
(60) Provisional application No. 63/680,170, filed on Aug. 7, 2024.

(51) Int. Cl. A61M 39/10; A61M 5/14; A61M 5/20; A61M 31/00

(52) U.S. Cl. CPC A61M 39/10; A61M 5/1413; A61M 5/20; A61M 31/00; A61M 2039/1077; A61M 2210/0618; A61M 2210/12

(57) ABSTRACT

An adapter for delivering medication from an autoinjector to a medical delivery system includes a hollow body and a sealing mechanism. The hollow body can define an internal cavity and can include an opening, a cutout, and a protrusion. The opening can be disposed at a first end of the hollow body. The opening can receive the autoinjector into the internal cavity of the hollow body. The cutout can be disposed at a second end of the hollow body opposite the first end. The protrusion can be disposed adjacent the cutout and can extend outwardly from the hollow body. The protrusion can have a port disposed within the protrusion. The protrusion can have a connection portion configured to interface with the medical delivery system. The sealing mechanism can be disposed within the hollow body adjacent the port.

### CROSS-REFERENCE TO RELATED APPLICATIONS

This application claims the benefit of U.S. Provisional Application No. 63/680,170, filed on Aug. 7, 2024. The entire disclosure of the above application is incorporated herein by reference.

### FIELD

The present technology relates to medical delivery systems, and, more particularly, to devices facilitating the administration of medications through various pathways using an autoinjector.

### INTRODUCTION

This section provides background information related to the present disclosure which is not necessarily prior art.

The administration of medication in an emergency and high-risk environment presents challenges that persist across various medical scenarios. In environments such as battlefield conditions with a military medic, an emergency situation with a paramedic and an EMS first responder, and an emergency room facilities treating a high-risk patient, a medical professional can face obstacles when attempting to deliver lifesaving intervention. The medical professional operating in the conditions must rely on parenteral drug administration through injection pathways including intravenous (IV), intramuscular (IM), or subcutaneous (SubQ) delivery methods. Each pathway requires equipment and expertise, creating a logistical burden that can compound the challenges of providing care in an unstable environment. In a battlefield scenario, the medic must carry multiple syringe-and-needle combinations to treat several patients, along with fragile glass vials containing drug products that require protection from damage in kinetic environments. The combination of syringes, needles, glass vials, and protective housing can increase the weight of the medical loadout and can complicate the number of items medics must manage during treatment delivery.

Limitations of certain medication or drug delivery methods create hazards for both patients and caregivers. An exposed needle can present inherent danger when not properly controlled. The exposed needle can additionally introduce contamination pathways into the IV system, while the high activation force required for auto-injectors poses concerns that can result in either dose loss or needle stick injuries to the medical professional. Time constraints in emergency medical scenarios further exacerbate the challenges. The interval between injury and drug administration can determine patient survival outcomes, yet syringe-and-needle and IV administration pathways require setup time that the patient may not have available. The complexity of the delivery method can demand trained medical professionals, creating a bottleneck in patient care when medical personnel are scarce. This can become particularly problematic in an on-site emergency or austere conditions where the number of available medical professionals restricts the ability to triage and simultaneously treat multiple patients. Incompatibility between an auto-injector and medical infrastructure can further complicate emergency care scenarios. Where the auto-injector cannot interface with IV bags or injection ports for intravenous drug delivery, this limits the versatility in situations where administering a systemic drug would be more appropriate than a localized injection. Such incompatibility forces the medical personnel to maintain separate systems for different administration routes, increasing equipment loads and reducing operational efficiency.

Accordingly, there is a continuing need for an adaptable and efficient drug delivery system that can interface with multiple administration pathways while reducing equipment burden and enabling rapid medication delivery by personnel with varying levels of medical training in high-risk environments.

### SUMMARY

In concordance with the instant disclosure, an adaptable and efficient drug delivery system that can interface with multiple administration pathways while reducing equipment burden and enabling rapid medication delivery by personnel with varying levels of medical training in high-risk environments, has surprisingly been discovered. The present technology includes articles of manufacture, systems, and processes that relate to the delivery of medication through an adaptable autoinjector capable of interfacing with various administration routes, including intravenous, intramuscular, and subcutaneous pathways. In this way, treatment efficiency can be enhanced in emergency medical scenarios.

In certain embodiments, an adapter for delivering medication from an autoinjector to a medical delivery system includes a hollow body and a sealing mechanism. The hollow body can define an internal cavity and can include an opening, a cutout, and a protrusion. The opening can be disposed at a first end of the hollow body. The opening can receive the autoinjector into the internal cavity of the hollow body. The cutout can be disposed at a second end of the hollow body opposite the first end. The protrusion can be disposed adjacent the cutout and can extend outwardly from the hollow body. The protrusion can have a port disposed within the protrusion. The protrusion can have a connection portion configured to interface with the medical delivery system. The sealing mechanism can be disposed within the hollow body adjacent the port.

In certain embodiments, a system for drug delivery to a patient can include an autoinjector and an adapter. The autoinjector can be configured for medication or drug delivery by intramuscular delivery. The adapter can be in fluid communication with the autoinjector and can enable the autoinjector to be used for the medication or drug delivery by one of intravenous delivery and nasal delivery.

In certain embodiments, a method for medication delivery to a patient can include providing an autoinjector and an adapter. The autoinjector can be configured for the drug delivery by intramuscular delivery. The adapter can be configured to interface with the autoinjector. The adapter can enable the autoinjector to be used for the drug delivery by one of intravenous delivery and nasal delivery. The adapter can be placed in fluid communication with the autoinjector. The medication or drug can be administered to the patient.

Further areas of applicability will become apparent from the description provided herein. The description and specific examples in this summary are intended for purposes of illustration only and are not intended to limit the scope of the present disclosure.

### DRAWINGS

The drawings described herein are for illustrative purposes only of selected embodiments and not all possible implementations and are not intended to limit the scope of the present disclosure.

FIG. 1 is a top perspective view of an adapter;

FIG. 2 is a front elevational view of the adapter;

FIG. 3 is a rear elevational view of the adapter;

FIG. 4 is a left-side elevational view of the adapter;

FIG. 5 is a top plan view of the adapter;

FIG. 6 is a right-side view of the adapter;

FIG. 7 is a bottom plan view of the adapter;

FIG. 8 is a cross-sectional, side elevational view of the adapter taken at section line 8-8 of FIG. 5;

FIG. 9 is a top perspective view of a system for drug delivery to a patient including the adapter and an auto-injector where the adapter is installed on the autoinjector;

FIG. 10 is cross-sectional, side elevational view of the adapter installed on the autoinjector taken at section line 10-10 of FIG. 9;

FIG. 11 is a perspective view of the system coupled to a medical delivery system, such as an intravenous line;

FIG. 12 is a cross-sectional, side elevational view of the adapter coupled to the medical delivery system, such as nasal atomization device;

FIG. 13 is a cross-sectional, side elevational view of the adapter coupled to the intravenous line taken at section line 13-13 of FIG. 11;

FIG. 14 is a schematic depicting a kit for medication delivery to a patient; and

FIG. 15 is a flowchart depicting a method medication delivery to a patient.

### DETAILED DESCRIPTION

The following description of technology is merely exemplary in nature of the subject matter, manufacture and use of one or more inventions, and is not intended to limit the scope, application, or uses of any specific invention claimed in this application or in such other applications as may be filed claiming priority to this application, or patents issuing therefrom. Regarding methods disclosed, the order of the steps presented is exemplary in nature, and thus, the order of the steps can be different in various embodiments, including where certain steps can be simultaneously performed, unless expressly stated otherwise. “A” and “an” as used herein indicate “at least one” of the item is present; a plurality of such items may be present, when possible. Except where otherwise expressly indicated, all numerical quantities in this description are to be understood as modified by the word “about” and all geometric and spatial descriptors are to be understood as modified by the word “substantially” in describing the broadest scope of the technology. “About” when applied to numerical values indicates that the calculation or the measurement allows some slight imprecision in the value (with some approach to exactness in the value; approximately or reasonably close to the value; nearly). If, for some reason, the imprecision provided by “about” and/or “substantially” is not otherwise understood in the art with this ordinary meaning, then “about” and/or “substantially” as used herein indicates at least variations that may arise from ordinary methods of measuring or using such parameters.

All documents, including patents, patent applications, and scientific literature cited in this detailed description are incorporated herein by reference, unless otherwise expressly indicated. Where any conflict or ambiguity may exist between a document incorporated by reference and this detailed description, the present detailed description controls.

Although the open-ended term “comprising,” as a synonym of non-restrictive terms such as including, containing, or having, is used herein to describe and claim embodiments of the present technology, embodiments may alternatively be described using more limiting terms such as “consisting of” or “consisting essentially of.” Thus, for any given embodiment reciting materials, components, or process steps, the present technology also specifically includes embodiments consisting of, or consisting essentially of, such materials, components, or process steps excluding additional materials, components or processes (for consisting of) and excluding additional materials, components or processes affecting the significant properties of the embodiment (for consisting essentially of), even though such additional materials, components or processes are not explicitly recited in this application. For example, recitation of a composition or process reciting elements A, B and C specifically envisions embodiments consisting of, and consisting essentially of, A, B and C, excluding an element D that may be recited in the art, even though element D is not explicitly described as being excluded herein.

Disclosures of ranges are, unless specified otherwise, inclusive of endpoints and include all distinct values and further divided ranges within the entire range. Thus, for example, a range of “from A to B” or “from about A to about B” is inclusive of A and of B. Disclosure of values and ranges of values for specific parameters (such as amounts, weight percentages, etc.) are not exclusive of other values and ranges of values useful herein. It is envisioned that two or more specific exemplified values for a given parameter may define endpoints for a range of values that may be claimed for the parameter. For example, if Parameter X is exemplified herein to have value A and also exemplified to have value Z, it is envisioned that Parameter X may have a range of values from about A to about Z. Similarly, it is envisioned that disclosure of two or more ranges of values for a parameter (whether such ranges are nested, overlapping or distinct) subsume all possible combination of ranges for the value that might be claimed using endpoints of the disclosed ranges. For example, if Parameter X is exemplified herein to have values in the range of 1-10, or 2-9, or 3-8, it is also envisioned that Parameter X may have other ranges of values including 1-9, 1-8, 1-3, 1-2, 2-10, 2-8, 2-3, 3-10, 3-9, and so on.

When an element or layer is referred to as being “on,” “engaged to,” “connected to,” or “coupled to” another element or layer, it may be directly on, engaged, connected or coupled to the other element or layer, or intervening elements or layers may be present. In contrast, when an element is referred to as being “directly on,” “directly engaged to,” “directly connected to” or “directly coupled to” another element or layer, there may be no intervening elements or layers present. Other words used to describe the relationship between elements should be interpreted in a like fashion (e.g., “between” versus “directly between,” “adjacent” versus “directly adjacent,” etc.). As used herein, the term “and/or” includes any and all combinations of one or more of the associated listed items.

Although the terms first, second, third, etc. may be used herein to describe various elements, components, regions, layers and/or sections, these elements, components, regions, layers and/or sections should not be limited by these terms. These terms may be only used to distinguish one element, component, region, layer or section from another region, layer or section. Terms such as “first,” “second,” and other numerical terms when used herein do not imply a sequence or order unless clearly indicated by the context. Thus, a first element, component, region, layer or section discussed below could be termed a second element, component, region, layer or section without departing from the teachings of the example embodiments.

Spatially relative terms, such as “inner,” “outer,” “beneath,” “below,” “lower,” “above,” “upper,” and the like, may be used herein for ease of description to describe one element or feature's relationship to another element(s) or feature(s) as illustrated in the figures. Spatially relative terms may be intended to encompass different orientations of the device in use or operation in addition to the orientation depicted in the figures. For example, if the device in the figures is turned over, elements described as “below” or “beneath” other elements or features would then be oriented “above” the other elements or features. Thus, the example term “below” can encompass both an orientation of above and below. The device may be otherwise oriented (rotated 90 degrees or at other orientations) and the spatially relative descriptors used herein interpreted accordingly.

As used herein, “medication” or “drug” can be defined as any medication or drug that can be administered via intramuscular delivery, intravenous delivery, subcutaneous delivery, nasal delivery, and combinations thereof. For example, “medication” can include a medication selected from the group of medications identified by tradenames consisting of Acthar, Actimmune, Apokyn, AquaMephyton, Aranesp, Arixtra, Avonex, Betaseron, Bravelle, Butorphanol, Byetta, Calcijex, Calcitonin, Caverject, Cetrotide, Chorionic Gonadotropin, Cimzia, Copaxone, Copegus, DDAVP, D.H.E-45, Delatestryl, Delestrogen, Depo-Estradiol, Depo-Provera 150, Depo-SubQ Provera 104, Depo-Testosterone, Desmopressin, Dihydroergotamine, Edex, Eligard, Enbrel, Epipen, Epogen, Exjade, Faslodex, Fertinex, Follistim, Forteo, Fragmin, Fuzeon, Ganirelix acetate, Genotropin, Gleevec, Glucagon, Gonal, Heparin, Humatrope, Humira, Imitrex, Increlex, Infergen, Innohep, Insulin, Intron A, iPlex, Ketorolac, Kestrone, Kineret, Kuvan, Leukine, Leuprolide Acetate, Lovenox, Lupron, Luveris, Medroxyprogesterone, Menopur, Methotrexate, Miacalcin, Muse, Neumega, Neulasta, Neupogen, Nexavar, Norditropin, Novarel, Nutropin, NuvaRing, Omnitrope, Orfadin, Ovidrel, Pegasys, Peg-Intron, Pregnyl, Procrit, Profasi, Progesterone, Pulmozyme, Raptiva, Rebetol, Rebif, Repronex, Revlimid, Ribasphere, Ribavirin, Saizen, Sandostatin, Sensipar, Serostim, Somatuline, Sprycel, Somavert, Stadol, Sumatriptan, Supprelin, Sutent, Symlin, Tarceva, Testosterone, Temodar, Tev-Tropin, Thalomid, Tobi, Tykerb, Vitamin B12, Vitamin D, Vitamin K, Xeloda, Zemplar, and Zorbtive.

The present technology improves the efficiency of administering medication by providing an autoinjector system that seamlessly integrates with existing medical infrastructure, allowing for rapid switching between intravenous, intramuscular, and subcutaneous delivery methods without the need for multiple distinct devices. The present technology can minimize the logistical burden of medical supply in an emergency situation by militating against the need to carry a separate system for intravenous and/or intramuscular administration. In this way, administration of medication can be optimized, thereby enhancing the capability of a medical responder to provide timely and effective care in an emergency situation.

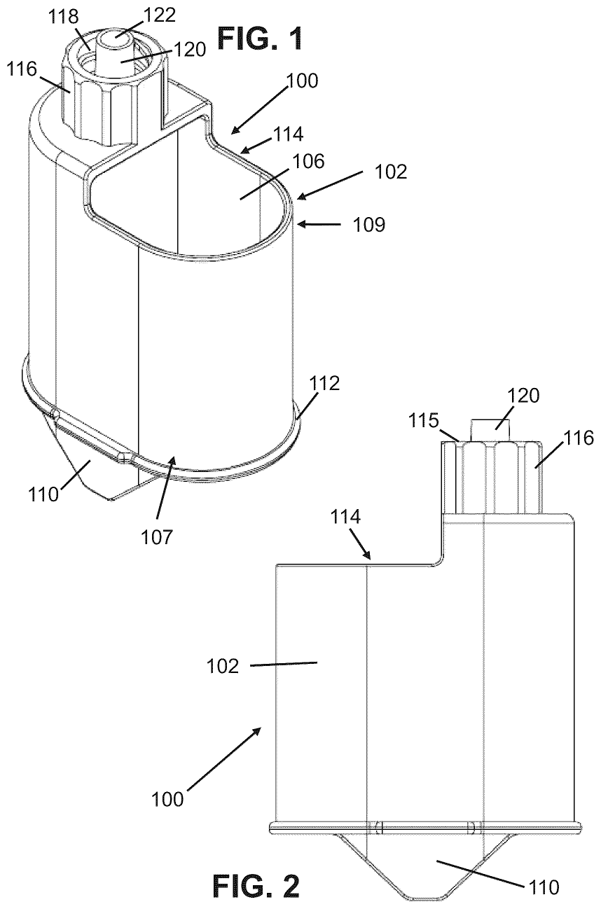

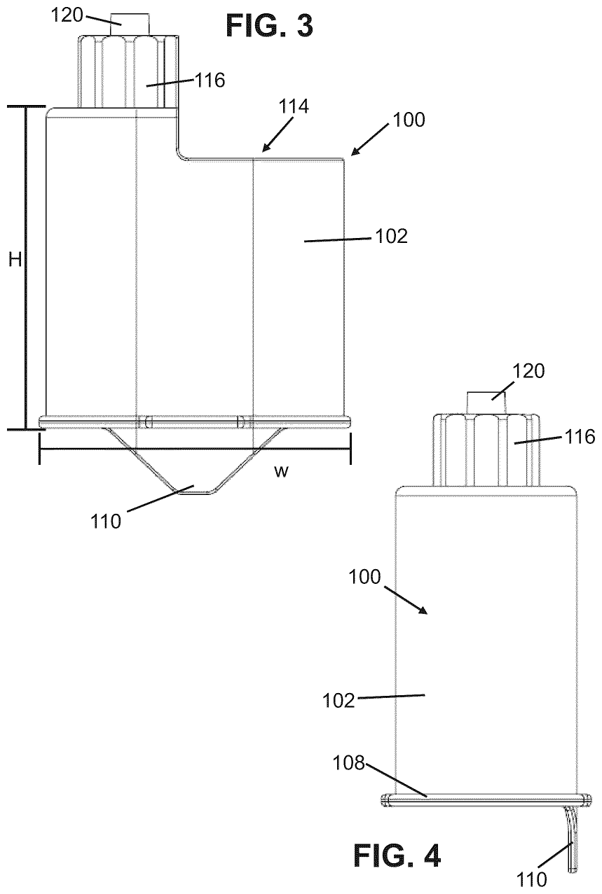

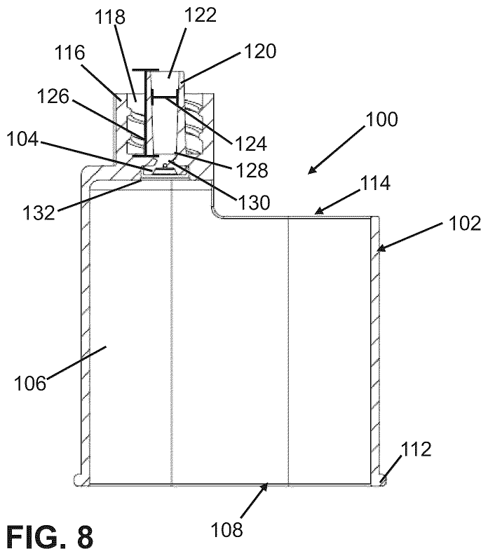

With reference now to FIGS. 1-13, an embodiment of an adapter 100 for delivering medication from an autoinjector 101 to a medical delivery system 103, is shown. In operation, the adapter 100 can be used with the autoinjector 101. The medical delivery system 103 can be coupled to the autoinjector 101 and a needle 105 from the autoinjector 101 can pass through the adapter 100 into the medical delivery system 103 to delivery medication from the autoinjector 101 to the medical delivery system 103. As shown in FIG. 8, the adapter 100 can include a hollow body 102 and a sealing mechanism 104.

It should be appreciated that the adapter 100 disclosed herein can be configured for use with various types of autoinjectors 101, including the medication administration device disclosed in U.S. patent application Ser. No. 17/203,728, filed Mar. 16, 2021, titled MINIATURIZED WEARABLE MEDICATION ADMINISTRATION DEVICE, which is incorporated herein by reference in its entirety, to provide enhanced versatility and expanded delivery options for a wearable medication system. Additionally, the medical delivery system 103 can include any medical delivery system capable of intramuscular delivery, intravenous delivery, subcutaneous delivery, nasal delivery, and combinations thereof. A skilled artisan can select an adapter 100 configured to operate with a suitable autoinjector 101 and a medical delivery system 103 within the scope of the present disclosure.

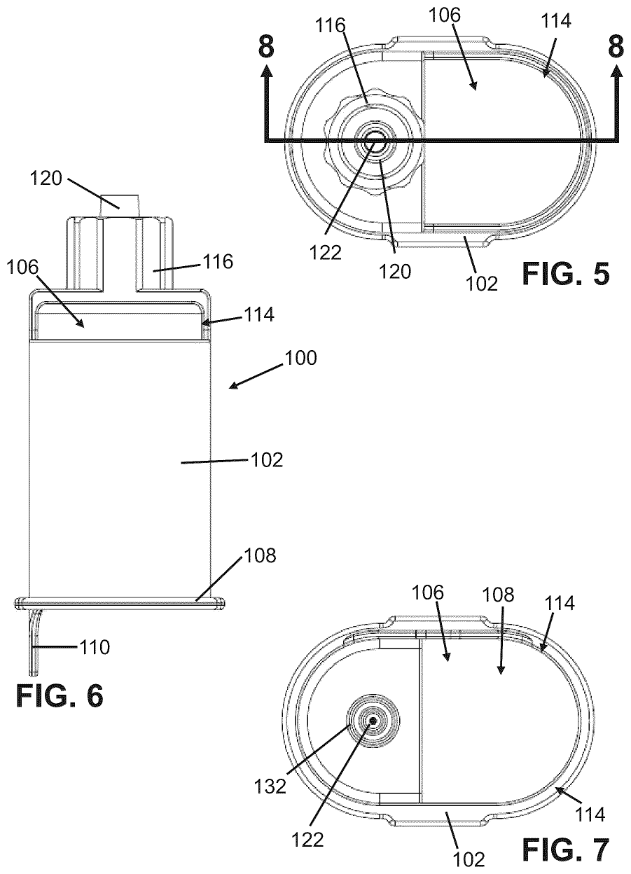

With reference to FIGS. 1 and 8, the hollow body 102 of the adapter 100 can define an internal cavity 106 that can be configured to receive the autoinjector 101 into the internal cavity 106 of the hollow body 102. The hollow body 102 can include an oblong cross-section, as shown in FIGS. 5 and 7. The oblong cross-section can provide an optimal geometric configuration for securely accommodating an autoinjector 101 of various sizes while maintaining structural integrity. The oblong cross section can offer an advantage by providing better grip surfaces and militating against rotational movement of the autoinjector 101 when inserted into the internal cavity 106 of the adaptor 102. The internal cavity 106 can be dimensioned to receive the autoinjector 101 while maintaining alignment for fluid communication between the autoinjector 101, the adapter 100, and the medical delivery system 103.

The hollow body 102 can be constructed from a biocompatible material suitable for medical applications, including medical-grade plastic. For example, the hollow body 102 can be formed from polycarbonate, acrylonitrile butadiene styrene (ABS), or polypropylene, which can offer the necessary strength, chemical resistance, and sterilization compatibility required for a medical device. Alternative materials can include medical-grade silicones for applications requiring flexibility, or metal alloys such as stainless steel for enhanced durability in reusable configurations. One or more materials included in the adapter 100 can be specifically selected to facilitate easy cleaning and sterilization within the medical context. A skilled artisan can select a suitable material for the adapter 100 within the scope of the present disclosure.

The hollow body 102 can have a height (H) and a width (W). The height (H) can be substantially greater than the width (W) of the hollow body 102, creating an elongated profile. The height-to-width ratio can promote a secure coupling between the autoinjector 101 and the adapter 100 by providing sufficient engagement length along the body of the autoinjector 101. The height of the adapter 100 can allow for better mechanical stability and can militate against the adapter 100 from becoming loose or disconnected during use. For example, the adaptor 100 can be configured and dimensioned to provide a friction-fit or snap fit arrangement when a portion of the autoinjector 101 is disposed within the internal cavity 106. The dimensional relationship of the adapter 100 can also provide adequate space for the internal cavity 106 while maintaining the structural integrity needed to withstand the forces generated during medication delivery.

The hollow body 102 can interact with the autoinjector 101 through an opening 108 disposed at a first end 107 of the hollow body 102 that receives the autoinjector 101 into the internal cavity 106 of the adapter 100. The adapter 100 can include a tab 110 that corresponds with a depression 105 on the exterior of the autoinjector 101, which can promote optimal alignment and further secure the autoinjector 101 within the adapter 100. The tab 110 and corresponding depression 105 can create a friction fit that militates against unwanted movement or rotation of the autoinjector 101 during use. In certain embodiments, the tab 110 can have a trapezoidal cross section which can correspond with a trapezoidal depression 105 on the exterior of the autoinjector 101. A skilled artisan can select a suitable tab 110 and depression 105 shape and size to optimize the friction fit between the autoinjector 101 and the adapter 100, allowing for customization based on specific autoinjector 101 and desired retention force. The geometric correspondence between the adapter 100 and the autoinjector 101 can ensure that the autoinjector 101 can only be inserted in the correct orientation, militating against misalignment that could compromise fluid communication or medication delivery.

The hollow body 102 can have a lip 112 that circumscribes the opening 108, which can allow the user to easily install the adapter 100 on the autoinjector 101 or remove the adapter 100 from the autoinjector 101. The lip 112 can provide a gripping surface that enables users to manipulate the adapter 100, which can be particularly important in an emergency medical scenario where quick and reliable operation is desired. The lip 112 can be sized to provide adequate leverage for the user while remaining slim enough to maintain a streamlined profile of the autoinjector 101 and adapter 100. The balance between functionality and form factor can ensure that the combined adapter 100 and autoinjector 101 remains portable and easy to handle while providing sufficient mechanical advantage for secure attachment and detachment. The lip 112 can distribute gripping forces evenly around the opening 108, reducing stress concentration that can lead to material failure and promoting consistent performance across multiple uses.

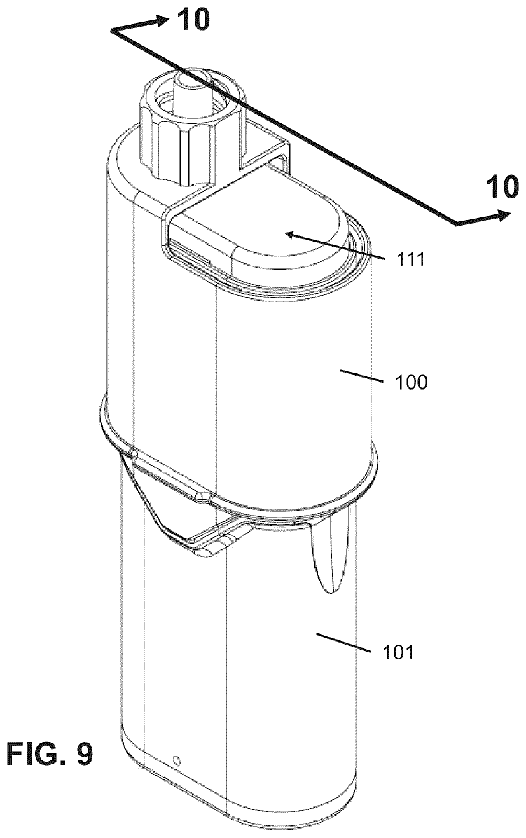

A cutout 114 of the hollow body 102 can be disposed at a second end 109 of the hollow body 102 opposite the first end 107 of the hollow body 102. The cutout 114 can be positioned opposite the opening 108 of the hollow body 102, creating an access point into the internal cavity 106. In operation where the adapter 100 is installed on the autoinjector 101, the cutout 114 can allow for a user to access an activation button 111 of the autoinjector 101 and dispense the medication within the autoinjector 101. As shown in FIG. 9, the shape and dimensions of the cutout 114 can correspond to the activation button 111 of the autoinjector 101. Additionally, the shape and dimensions of the cutout 114 can balance functionality with structural integrity. The cutout 114 can be only a portion of a side of the hollow body 102 rather than an entire side, which can assist with maintaining the structural strength of the adapter 100 while preserving a slim profile. The cutout 114 can allow sufficient space for other components, such as a protrusion 116, to be disposed on the first end 107 with the cutout 114 without compromising the overall dimensions of the adapter 100. By limiting the cutout 114 to only a portion of the hollow body 102, the adapter 100 can maintain material thickness around the cutout 114 perimeter to provide structural support and integrity of the adaptor 100 during use.

As shown in FIG. 1, the hollow body 102 can include the protrusion 116. The cutout 114 can be positioned adjacent the cutout 114. The placement of the protrusion 116 on the hollow body 102 can allow the user to depress the activation button 111 of the autoinjector 101 and simultaneously visualize that the medication is exiting the autoinjector 101 through the adapter 100 into the medical delivery system 103. The visual confirmation can be particularly important in a medical scenario where verification of medication flow is important for ensuring successful medication delivery. The positioning of the protrusion 116 relative the cutout 114 can enable real-time monitoring of the medication transfer process, allowing the user to confirm that the adapter 100 and the autoinjector 101 are functioning correctly and that the medication is being delivered to the intended medical delivery system 103.

The protrusion 116 can extend outwardly from the hollow body 102, creating an interface point for connection of the autoinjector 101 to the medical delivery system 103. The outward extension of the protrusion 116 can position the connection point between the autoinjector 101 and the medical delivery system 103 away from the hollow body 102 of the adapter 100, facilitating access and manipulation during use. In operation, the protrusion 116 can be aligned with the needle hub of the autoinjector to allow for the needle 105 of the autoinjector 101 to pass through the protrusion 116. The protrusion 116 can be configured to receive and guide the needle 105 from the autoinjector 101, ensuring optimal positioning and militating against misalignment that could interfere with the medication delivery process. The needle-receiving capability of the protrusion 116 can allow the autoinjector 101 to interface with various medical delivery systems 103 while maintaining the integrity of the medication pathway throughout the medication administration process.

As shown in FIG. 8, the protrusion 116 can include a connection portion 118. The connection portion 118 can provide a versatile and a secure connection between the adapter 100 and the medical delivery system 103 through multiple interface options. The connection portion 118 of the protrusion 116 can utilize any type of coupling means to achieve secure and reliable attachment to the medical delivery system 103, including a threaded connection, a snap fit connection, a Luer lock connection, a bayonet connection, a compression fitting, a quick-disconnect coupling, a friction fit connection, or a magnetic coupling system. A skilled artisan can select a suitable connection portion 118 within the scope of the present disclosure.

As shown in the figures, the protrusion 116 can include a port 120 disposed within the protrusion 116 which establishes fluid communication between the autoinjector 101 and the medical delivery system 103. The port 120 can disposed through the protrusion 116 and into the hollow body 102. In certain embodiments, the port 120 can have a circular cross section to correspond with the medical delivery system 103, which can provide optimal compatibility with various medical connectors and tubing systems used in medical delivery systems 103. Advantageously, the circular cross section can promote uniform flow characteristics and can minimize turbulence or flow restrictions that could affect medication delivery rates or accuracy. The geometric configuration can also provide a consistent sealing surface when interfacing with the medical delivery system 103, thereby optimizing coupling with the medical delivery system 103.

As shown in FIG. 2, the port 120 can extend beyond a distal surface 115 of the protrusion 116, which can provide additional engagement length for interfacing with the medical delivery system 103. The additional engagement length can facilitate a more secure and stable connection between the adapter 100 and the medical delivery system 103 that can withstand the forces and pressures encountered during medication delivery. The port 120 can also facilitate more direct visual confirmation of the connection between the adapter 100 and the medical delivery system 103, allowing the user to verify that the medical delivery system 103 is optimally seated and engaged with the adapter 100. Additionally, the port 120 extending beyond the protrusion 116 can provide clearance between the protrusion 116 and the connected medical delivery system 103, militating against interference that could compromise the connection integrity or make disconnection difficult.

The port 120 can have a channel 122 that serves as the pathway for the needle 105 of the autoinjector 101 to move through the adapter 100 and into the medical delivery system 103 to allow for the medication flow through the needle 105 into the medical delivery system 103. The channel 122 can have a varied diameter along a length 126 of the channel 122, which can provide control over the flow characteristics of the medication as the medication passes from the autoinjector 101 to the medical delivery system 103. In certain embodiments, the channel 122 can have a variable diameter 124, such as where an interior end 128 of the channel 122 can be smaller in diameter compared to other portions of the channel 122, thereby creating a flow restriction that can affect a rate of medication delivery. As medication moves out of the needle 105 of the autoinjector 101 to the medical delivery system 103, the rate of flow can change based on the difference in diameter between various sections of the channel 122. The variable diameter 124 can allow for precise flow rate management based on the specific requirements of different medications or delivery scenarios. The variable diameter 124 can create a venturi effect or flow restriction that can either accelerate or decelerate the medication flow depending on the specific diameter relationships along the length 126 of the channel 122.

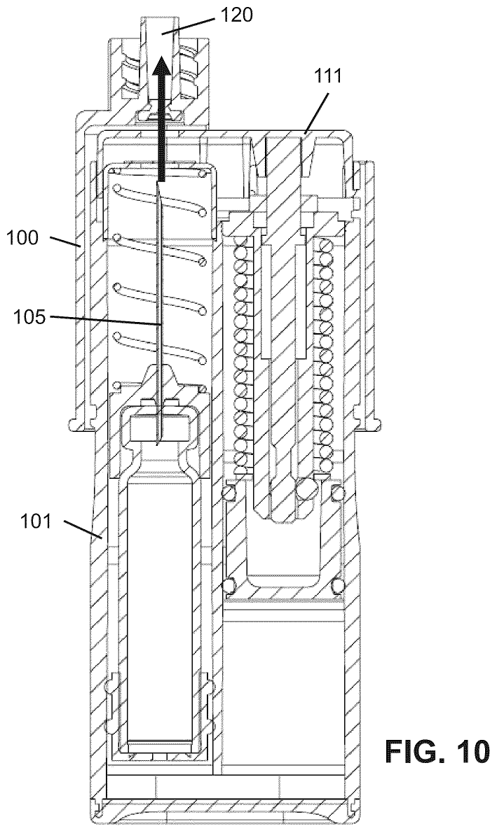

With reference to FIG. 10, the internal cavity 106 can be open to the port 120 such that the needle 105 of the autoinjector 101 can exit the autoinjector 101 and move through the port 120 and into the medical delivery system 103 for delivery. As shown in FIG. 8, the internal cavity 106 can have an indentation 132 that abuts the interior of the channel 122 of the port 120, which creates a passageway 130 between the internal cavity 106 and the port 120. The passageway 130 between the internal cavity 106 and the port 120 can allow the needle 105 of the autoinjector 101 to extend beyond a housing of the autoinjector 101 and establish fluid communication with the connected medical delivery system 103. The indentation 132 can serve as a transition zone between the larger internal cavity 106 and the smaller port 120.

The adapter 100 can include a sealing mechanism 104 disposed in the indentation 132 as an intermediary between the internal cavity 106 and the port 120. The placement of the sealing mechanism 104 within the indentation 132 can allow the sealing mechanism 104 to control access to the port 120 and the medical delivery system 103 while maintaining the sterility of the passageway 130 when the adapter 100 is not in use. The sealing mechanism 104 can help to militate against contamination of the needle 105 of the autoinjector 101 when the adapter 100 is not in use to connect the autoinjector 101 to the medical delivery system 103, thereby maintaining the sterility and integrity of the medication delivery system 103.

The sealing mechanism 104 can function through various operational principles depending on the requirements of the medical delivery system 103. The sealing mechanism 104 can operate as a self-actuating seal that automatically closes when external pressure is removed, such as when the medical delivery system 103 is disconnected from the adapter 100. The sealing mechanism 104 can utilize elastic deformation to create a barrier across the interior end 128 of the port 120, militating against contaminants from entering the passageway 130. Where the medical delivery system 103 is connected to the adapter 100, the sealing mechanism 104 can be displaced or compressed to allow fluid communication, and upon disconnection, the sealing mechanism 104 can return to a sealing position to maintain sterility.

The sealing mechanism 104 can have various shapes optimized for effective sealing performance. The sealing mechanism 104 can have a disc shape that spans the indentation 132 and the opening of the port 120, providing a flat sealing surface that can be easily displaced during connection. Alternatively, the sealing mechanism 104 can have a conical or dome shape that can provide progressive sealing as the sealing mechanism 104 deforms under pressure. The sealing mechanism 104 can also have a slit or cross-cut configuration that allows for penetration by the needle 105 of the autoinjector 101 while maintaining sealing capability around a perimeter of the sealing mechanism 104. A skilled artisan can select a suitable sealing mechanism 104 within the scope of the present disclosure.

The sealing mechanism 104 can be constructed from various materials that provide the necessary sealing properties and biocompatibility for medical applications. The sealing mechanism 104 can be made from medical-grade elastomers such as silicone rubber, which can provide sealing characteristics, chemical resistance, and sterilization compatibility. Alternative materials can include thermoplastic elastomers, natural rubber compounds, or synthetic rubber materials that offer appropriate flexibility and sealing performance, for example. The material selection can consider factors such as chemical compatibility with medications, sterilization requirements, durability for multiple uses in reusable configurations, and regulatory compliance for medical device applications. The material can also be selected to provide appropriate hardness and compression characteristics to ensure reliable sealing while allowing for easy penetration by the needle 105 of the autoinjector 101 during use.

It should be appreciated that the hollow body 102 of the adapter 100 can be manufactured using various production methods suitable for medical device fabrication. Injection molding can be employed as a manufacturing method, which can be advantageous for high-volume production of the hollow body 102. The manufacturing process can utilize medical-grade thermoplastic materials in heated molds to create precise, repeatable components with dimensional accuracy and surface finish. In certain embodiments, the hollow body 102 can be formed using a 3D printing process, allowing for flexibility and rapid iteration capabilities.

The present disclosure provides a system 200 for medication delivery to a patient. The system 200 can include the adapter 100, as described herein, and the autoinjector 101. The system 200 can be used to enable the autoinjector 101 to adapt for medication delivery the medical delivery system 103, such as by either intravenous delivery or nasal delivery. In operation, the system 200 can enable intravenous delivery where a medication needs to be administered directly into the bloodstream for rapid systemic effects, or nasal delivery for a medication that benefits from rapid absorption through the nasal mucosa, which can be advantageous for patients who are unconscious or unable to swallow oral medications.

The system 200 operates through the integration of the needle-based autoinjector 101 with the adapter 100, as described herein. The hollow body 102 of the adapter 100 can receive the autoinjector 101 through the opening 108. The autoinjector 101 can be positioned within the internal cavity 106 such that the needle 105 of the autoinjector 101 aligns with the protrusion 116, allowing the needle 105 to pass through the port 120 of the adapter 100 and establish fluid communication with the medical delivery system 103 connected via the connection portion 118. The tab 110 can correspond with the depression of the autoinjector 101, ensuring proper orientation, and creating a friction fit and/or a snap fit that militates against unwanted movement or rotation of the autoinjector during use.

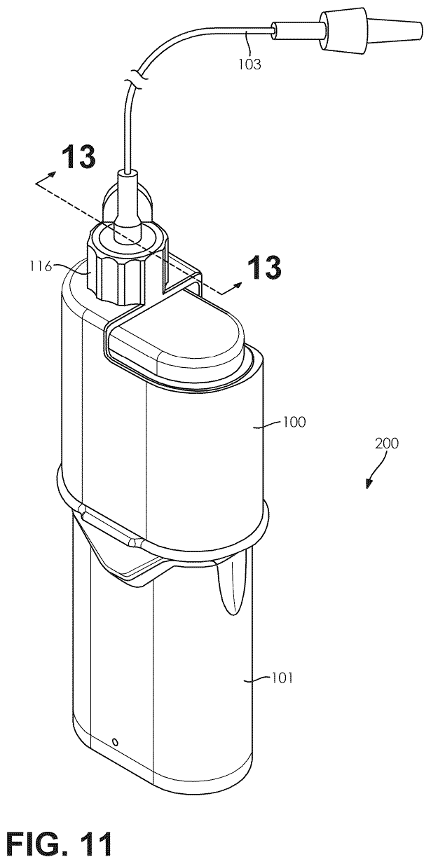

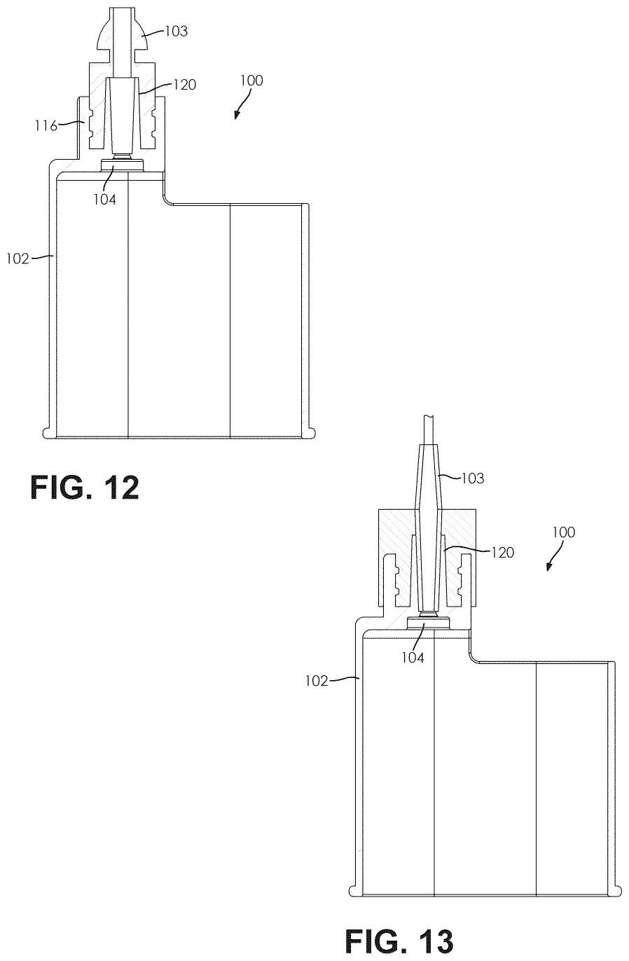

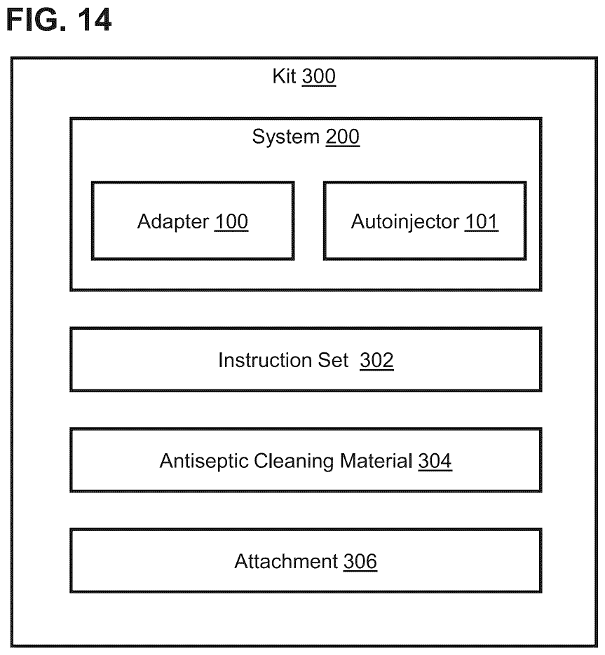

The present disclosure provides a kit 300 for medication delivery to a patient, shown generally in FIG. 14. The kit 300 can include the autoinjector 101 configured for medication delivery by intramuscular delivery and the adapter 100 configured to be in fluid communication with the autoinjector 101, enabling the autoinjector 101 to be used for medication delivery by either intravenous delivery or nasal delivery. The kit 300 can include multiple attachment 306 configurations to provide versatility in drug delivery methods, such as intravenous delivery or nasal delivery. The attachment in the kit 300 can be one of a plurality of attachments 306, which includes an attachment for intravenous delivery, as shown in FIGS. 11 and 13, and an adapter for nasal delivery, as shown in FIG. 12. The attachment 306 can allow for the customization of the drug delivery method according to specific medical requirements and patient preference. It should be appreciated that the connection portion 118 of the adapter 100 can couple the adapter 100 to any attachment 306 to facilitate delivery of the medication through any desired medical delivery system 103.

The kit 300 can further include an instruction set 302 for assembling the system 200 for medication delivery. The instruction set 302 can provide clear and concise guidance on how to assemble and use the system 200, promoting effective use of the system 200. The instruction set 302 can detail the optimal selection of adapter 100 for a specific delivery pathway, the optimal assembly procedure for placing the adapter 100 in fluid communication with the autoinjector 101, and the optimal method for connecting the assembled system 200 to various medical delivery systems 103.

The kit 300 can further include an antiseptic cleaning material 304. The antiseptic cleaning material 304 can be used to clean the various components of the system 200, such as the adapter 100, before and after use. The antiseptic cleaning material 304 can be particularly useful for the reusable adapter 100 configuration where sterilization between uses is desired to militate against cross-contamination and infection.

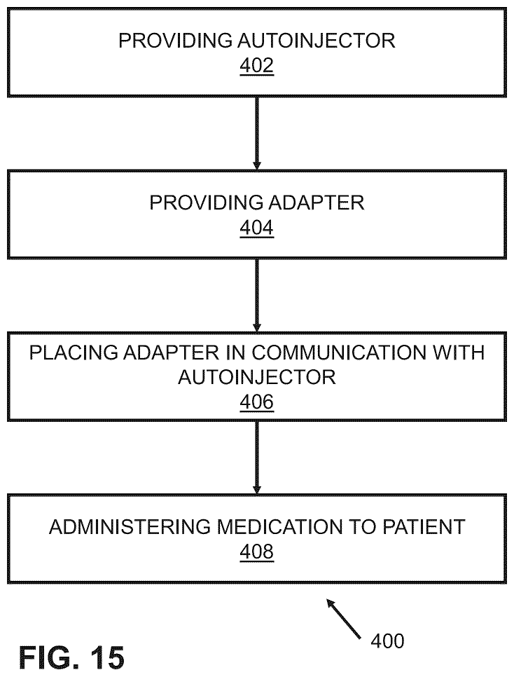

A method 400 for medication delivery to a patient is also provided, as shown generally in FIG. 15. The method 400 can include a step 402 of providing the autoinjector 101, as described herein. A skilled artisan can select any autoinjector 101 for use in the method 400. In a step 404, the method 400 can include providing the adapter 100 as described herein. The adapter 100 can be configured to interface with the autoinjector 101 such that the adapter 100 enabling the autoinjector 101 to be used for the medication delivery by one of intravenous delivery and nasal delivery. The method 400 can include placing the adapter 100 in communication with the autoinjector 101 in a step 406. In a step 408, the medication can be administered to the patient.

The adapter 100, system 200, kit 300, and method 400 of the present disclosure provide advantages in a medical care and drug delivery applications. The adapter 100 can enable versatility by allowing the autoinjector 101 to interface with multiple delivery pathways, including intravenous, intramuscular, and nasal routes, thereby militating against the need for multiple distinct devices and reducing the logistical burden on medical personnel. The system 200 can enhance operational efficiency by providing rapid switching between different administration routes without requiring extensive setup or training, which can enhance the capability of a medical responder to provide timely and effective care in a medical situation. The kit 300 can offer the medical professional flexibility with multiple adapter 100 configurations, instruction sets, and cleaning materials, ensuring that all components are readily available for a diverse medical scenario while maintaining the standards of hygiene and sterility. The method 400 can streamline a drug delivery procedure by simplifying the administration process and enabling medical personnel to safely administer the medication, optimizing the medical response.

### EXAMPLES

The following examples demonstrate embodiments of the present disclosure in use. The examples are provided for illustrative purposes only and should not be construed as limiting the scope of the present disclosure. It will be appreciated by those skilled in the art that various modifications, alternatives, and variations of the example can be made without departing from the scope of the present disclosure.

### Example 1: Intravenous Drug Delivery

In one embodiment, the system 200 can be utilized for intravenous drug delivery in an emergency medical scenario. A medical professional selects the autoinjector 101 pre-filled with a medication, such as an anticoagulant, and attaches the adapter 100 for intravenous delivery. The adapter 100, equipped with a threaded connection portion 118, can be securely connected to a medical delivery system 103, such as an existing intravenous line, set up on the patient. The setup allows the medication to be administered directly into the bloodstream of the patient, providing rapid systemic effects for the treatment of the patient.

The adapter 100 can include the sealing mechanism 104 that militates against any contamination of the medication during the transfer from the autoinjector 101 to the intravenous line 103. As the medical professional activates the autoinjector 101, the adapter 100 can provide tactile and audible feedback confirming a secure connection between the adapter 100 and the medical delivery system 103, thus minimizing the risk of accidental misadministration or dosage errors. Following the successful administration of the medication, the adapter 100 can be easily disconnected from the intravenous line 103 and disposed of or sterilized.

### Example 2: Nasal Drug Delivery

In another embodiment, the system 200 can be adapted for nasal drug delivery, which can be useful for administering medication that benefits from rapid absorption through the nasal mucosa. A healthcare provider selects the autoinjector 101, which can be pre-filled with a medication suitable for nasal administration, such as a fast-acting pain reliever or an anti-seizure medication. The provider attaches a nasal delivery attachment 306, which can fit comfortably into the nostril of the patient and ensure that the medication is dispersed within the nasal cavity.

Where the adaptor 100 is attached to the autoinjector 101, the provider can position the nasal end of the adapter 100 into the nostril of the patient and activate the autoinjector 101. The medication can be delivered as a fine mist, optimized for rapid absorption through the nasal passages.

After the administration is complete, the nasal adapter can be removed from the autoinjector 101 and either disposed of or cleaned for reuse. The method of nasal medication delivery is not only efficient but also minimizes the discomfort often associated with nasal administration, improving the patient experience and reducing stress for both the patient and the healthcare provider.

Example embodiments are provided so that this disclosure will be thorough, and will fully convey the scope to those who are skilled in the art. Numerous specific details are set forth such as examples of specific components, devices, and methods, to provide a thorough understanding of embodiments of the present disclosure. It will be apparent to those skilled in the art that specific details need not be employed, that example embodiments may be embodied in many different forms, and that neither should be construed to limit the scope of the disclosure. In some example embodiments, well-known processes, well-known device structures, and well-known technologies are not described in detail. Equivalent changes, modifications and variations of some embodiments, materials, compositions and methods can be made within the scope of the present technology, with substantially similar results.

What is claimed is:

1. An adapter for delivering medication from an autoinjector to a medical delivery system, comprising:
a hollow body defining an internal cavity, the hollow body having
an opening disposed at a first end of the hollow body for receiving the autoinjector into the internal cavity of the hollow body,
a cutout disposed at a second end of the hollow body opposite the opening of the hollow body,
a protrusion disposed adjacent the cutout and extending outwardly from the hollow body, the protrusion having a port disposed therethrough into the hollow body, the protrusion having a connection portion configured to interface with the medical delivery system; and
a sealing mechanism disposed within the hollow body adjacent the port.

2. The adapter of claim 1, wherein the hollow body includes a height and a width, the height being greater than the width.

3. The adapter of claim 1, wherein the hollow body includes a lip that circumscribes the opening.

4. The adapter of claim 1, wherein the hollow body includes a tab that corresponds with a depression on an exterior of the autoinjector.

5. The adapter of claim 1, wherein the adapter is configured to snap fit onto the autoinjector.

6. The adapter of claim 1, wherein the cutout is formed in a portion of the second end of the hollow body.

7. The adapter of claim 1, wherein the protrusion is disposed on the second end of the hollow body.

8. The adapter of claim 1, wherein the protrusion extends outwardly from the hollow body to position a connection point away from the hollow body.

9. The adapter of claim 1, wherein the port extends beyond an distal surface of the protrusion.

10. The adapter of claim 1, wherein the port has a channel having a varied diameter.

11. The adapter of claim 10, wherein an interior end of the channel is smaller in diameter compared to a remainder of the channel.

12. The adapter of claim 10, wherein the hollow body includes an indentation, and the sealing mechanism is disposed in the indentation and abuts the channel of the port.

13. The adapter of claim 1, wherein the connection portion includes a member selected from a group consisting of a snap fit connection, a bayonet connection, a compression fitting, a quick-disconnect coupling, a friction fit connection, and a magnetic coupling system.

14. The adapter of claim 1, wherein the sealing mechanism includes as a self-actuating seal.

15. A system for drug delivery to a patient, comprising:
an autoinjector configured for the drug delivery by intramuscular delivery; and
the adapter of claim 1,
wherein the adapter is in fluid communication with the autoinjector and enables the autoinjector to be used for the drug delivery the medical delivery system.

16. The system of claim 15, wherein the autoinjector is needle-based.

17. A method for drug delivery to a patient, the method comprising:
providing an autoinjector configured for the drug delivery by intramuscular delivery;
providing the adapter of claim 1, the adapter configured to interface with the autoinjector, enabling the autoinjector to be used for the drug delivery by one of intravenous delivery and nasal delivery;
placing the adapter in fluid communication with the autoinjector; and
administering a drug to the patient.

18. The method of claim 17, wherein the autoinjector is needle-based.

19. A kit for medication delivery to a patient, comprising:
an autoinjector configured for the drug delivery by intramuscular delivery;
the adapter of claim 1; and
an attachment configured to couple to the adapter.

20. The kit of claim 19, wherein the attachment includes at least one of an intravenous delivery attachment and a nasal delivery attachment.
